(19) United States
(12) Patent Application Publication
(10) Pub. No.: US 2025/0387347 A1
(43) Pub. Date: Dec. 25, 2025

(54) VITAMIN A AND D SOFT CAPSULE AND PREPARATION METHOD THEREFOR

(71) Applicant: SHANDONG DYNE MARINE BIOPHARMACEUTICAL CO., LTD, Rongcheng (CN)

(72) Inventors: Jie YANG, Rongcheng (CN); Shuwang HE, Rongcheng (CN); Shiqiang YAN, Rongcheng (CN); Longjiang WANG, Rongcheng (CN); Kun CAI, Rongcheng (CN); Xueping WU, Rongcheng (CN); Wensheng WANG, Rongcheng (CN); Huihui SUN, Rongcheng (CN); Xing HU, Rongcheng (CN)

(73) Assignee: SHANDONG DYNE MARINE BIOPHARMACEUTICAL CO., LTD, Rongcheng (CN)

(21) Appl. No.: 19/244,962

(22) Filed: Jun. 20, 2025

(30) Foreign Application Priority Data
Jun. 19, 2024 (CN) 202410793315.X

(51) Int. Cl. A61K 31/07; A61K 9/48; A61K 31/592; A61K 31/593

(52) U.S. Cl. CPC A61K 31/07; A61K 9/4825; A61K 9/4833; A61K 31/592; A61K 31/593

(57) ABSTRACT

The present disclosure belongs to the technical field of pharmaceutical preparations, and specifically relates to a vitamin A and D soft capsule and a preparation method therefor. In the present disclosure, it is found unexpectedly through research that a CGKRK peptide, used as an anti-aging agent and added into a capsule wall material with gelatin as a matrix, has a better anti-aging effect on the gelatin than a single amino acid, and based on a lower addition amount, can greatly alleviate the problems of soft capsule disintegration, delayed dissolution and a decreased dissolution rate, thereby improving the stability and bioavailability of a vitamin A and D soft capsule preparation. Meanwhile, the vitamin A and D soft capsule preparation has a simple and practicable preparation method and inexpensive and readily available raw materials, and is extremely suitable for industrial mass production, thus having a good practical application value.

### CROSS-REFERENCE TO RELATED APPLICATIONS

The present disclosure claims priority to Chinese Patent Application No. 202410793315.X, filed to China National Intellectual Property Administration on Jun. 19, 2024, entitled “VITAMIN A AND D SOFT CAPSULE AND PREPARATION METHOD THEREFOR”, the entire contents of which are incorporated herein by reference and constitute a part of the present disclosure for all purposes.

### FIELD OF TECHNOLOGY

The present disclosure belongs to the technical field of pharmaceutical preparations, and specifically relates to a vitamin A and D soft capsule and a preparation method therefor.

### BACKGROUND

The information disclosed in the background section is merely intended to increase understanding of the general background of the present disclosure, and is not necessarily taken as an acknowledgment or implication in any form that the information constitutes the prior art that has already become known to those of ordinary skill in the art.

Vitamin A and D supplements are used for preventing and treating deficiencies of vitamins A and D, such as rickets, nyctalopia, and infantile tetany. Soft capsules are a common dosage form for vitamin A and D preparations. Considering raw materials and process costs, most of commercially available vitamin A and D soft capsules adopt gelatin as a matrix of a capsule wall material. The gelatin is a hydrophilic macromolecule derived from collagen in connective tissues such as animal skin and bones, and includes main components such as proteins composed of various amino acids. During long-term storage, especially under high-temperature and high-humidity conditions, the gelatin is prone to having an aging phenomenon, and main reasons include self-oxidative crosslinking of amino acids (primarily arginine and lysine) in gelatin molecules and crosslinking with low-molecular-weight aldehydes introduced from the outside, leading to delayed disintegration of the gelatin capsule wall material and a decreased dissolution rate, thereby affecting drug absorption in the body.

To address the aging problem of the gelatin, various amino acids have been proven to have improvement effects. For example, as disclosed respectively in prior applications CN101711875A and CN101953815A of the applicant, in capsule skin containing 37-43% of the gelatin, 1.5-3% of lysine is added to solve the problem of a prolonged disintegration time caused by the gelatin aging, and a combination of fumaric acid and L-lysine is used as an antioxidant to inhibit aging of the capsule skin, wherein a weight ratio of the fumaric acid to the L-lysine is 1:10 to 1:30. According to He Yulian, et al. (Improvement of Disintegration Time of Black Wolfberry Extract Soft Capsules [J]. Food Industry, 2021(002):042.), impacts of glycine, L-cysteine and arginine that are added into a capsule shell and inclusions on the disintegration time of a black wolfberry extract soft capsule are studied, and effects of the three amino acids in alleviating a disintegration problem is ranked as follows: L-cysteine>glycine>arginine. However, the level of resisting the aging of the capsule wall material in the aforementioned prior art still needs to be further improved.

### SUMMARY

In view of the shortcomings in the prior art, an objective of the present disclosure is to provide a vitamin A and D soft capsule and a preparation method therefor. Specifically, in the present disclosure, it is found unexpectedly through research that a CGKRK peptide, used as an anti-aging agent and added into a capsule wall material with gelatin as a matrix, has a better anti-aging effect on the gelatin than a single amino acid, and based on a lower addition amount, can greatly alleviate the problems of soft capsule disintegration, delayed dissolution and a decreased dissolution rate, thereby improving the stability and bioavailability of a vitamin A and D soft capsule preparation. Based on the above research findings, the present disclosure is completed.

To achieve the above technical objective, the present disclosure provides the following technical solutions.

In a first aspect of the present disclosure, use of a CGKRK peptide as an anti-aging agent in preparation of a vitamin A and D soft capsule is provided.

Specifically, the use includes improving aging resistance of a capsule wall material of the vitamin A and D soft capsule to alleviate the problems of soft capsule disintegration, delayed dissolution and a decreased dissolution rate, thereby improving the stability and bioavailability of a vitamin A and D soft capsule preparation.

Raw materials of the capsule wall material of the vitamin A and D soft capsule may include gelatin.

In a second aspect of the present disclosure, a vitamin A and D soft capsule is provided, which includes a capsule wall material and a capsule core medicine liquid, and in parts by weight, a mass ratio of raw materials of the capsule wall material to the capsule core medicine liquid is 1:(1-3), and is further 1:(1-2), such as 1:1.5.

The raw materials of the capsule wall material include gelatin, a plasticizer, a CGKRK peptide, and water; and the capsule core medicine liquid includes vitamin A, vitamin D, and an oil solvent.

In a third aspect of the present disclosure, a method for preparing the aforementioned vitamin A and D soft capsule is provided, which includes the following steps:
S1. mixing and dissolving the other components excluding the gelatin and the CGKRK peptide in the raw materials of the capsule wall material under heating to obtain a mixture;
S2. swelling the gelatin in water, adding the swelled gelatin into the mixture prepared in the step S1, then adding the CGKRK peptide, performing even mixing and melting to prepare a gelatin solution while maintaining a heating temperature in the step S1, removing air bubbles in the gelatin solution, and then subjecting the gelatin solution to discharging, filtration and standing for later use;
S3. preparing the gelatin solution prepared in the step S2 into a gelatin strip;
S4. dissolving the vitamins A and D in the oil solvent to prepare the capsule core medicine liquid; and
S5. subjecting an effective amount of the capsule core medicine liquid and the gelatin strip prepared in the step S3 to pelleting to obtain the vitamin A and D soft capsule.

### Beneficial Technical Effects of One or More of the Above Technical Solutions

According to the above technical solutions, it is proposed for the first time that the CGKRK peptide, as the anti-aging agent and added into the capsule wall material with the gelatin as the matrix, has a better anti-aging effect on the gelatin than a single amino acid, and based on a lower addition amount, can greatly alleviate the problems of soft capsule disintegration, delayed dissolution and a decreased dissolution rate, thereby improving the stability and bioavailability of a vitamin A and D soft capsule preparation.

Meanwhile, the above vitamin A and D soft capsule preparation has a simple and practicable preparation method and inexpensive and readily available raw materials, and is extremely suitable for industrial mass production, thus having a good practical application value.

### DETAILED DESCRIPTION

It should be pointed out that the following detailed descriptions are all exemplary and are intended to provide further illustration of the present disclosure. Unless otherwise specified, all technical terms and scientific terms used herein have the same meanings as commonly understood by those of ordinary skill in the art to which the present disclosure belongs.

It should be noted that the terms used herein are only for describing specific embodiments and are not intended to limit exemplary embodiments of the present application. As used herein, unless otherwise expressly stated in the context, singular forms are also intended to include plural forms. In addition, it should also be understood that when the terms “include” and/or “comprise” are used in the specification, the terms indicate features, steps, operations, devices, components and/or combinations thereof.

Unless otherwise defined herein, all the technical terms and scientific terms used herein have the same meanings as commonly understood by those of ordinary skill in the art. Although methods and materials similar or equivalent to those described herein can also be used in the practice of the present disclosure, preferred methods and materials are described herein. Additionally, as used herein, the term “a”, “an”, or “one” includes pluralities or multiples, unless clearly indicated otherwise in the context. Numerical ranges throughout the entire application document include any subranges and any numerical values incremented by smallest subunits of given values within the ranges. Unless explicitly stated otherwise, numerical values throughout the entire application document represent approximate measurements or limitations of ranges of the embodiments including slight deviations from the given values, having values approximately mentioned, and having precise values mentioned. Except for detailed description of finally provided working examples, all numerical values of parameters (e.g., quantities or conditions) in the present application document (including the appended claims) should be understood as being modified by the term “about” in all cases, regardless of whether the “about” actually appears before the numerical values. The “about” indicates that a stated numerical value is allowed to have slight imprecision (have certain approximate precision to the value; approximately or reasonably approximate to the value; or approximate to the value). When the imprecision provided by the “about” is not understood in the ordinary sense in the art, the “about” used herein at least indicates variations that can be produced by common methods of measuring and using these parameters. For example, the “about” may include the variation of less than or equal to 10%, less than or equal to 5%, less than or equal to 4%, less than or equal to 3%, less than or equal to 2%, less than or equal to 1%, or less than or equal to 0.5%.

As used herein, unless clearly indicated otherwise in the context, all percentages are calculated by weight.

As used herein, a “soft capsule” refers to a dosage form prepared by sealing certain amounts of drugs, active ingredients, nutrients and herbal extracts as well as suitable auxiliary materials in a spherical, oval, or other-shaped soft capsule wall material.

As used herein, the term “gelatin” refers to a product obtained by partial hydrolysis of collagen contained in animal skin, bones, tendons, and ligaments. In one embodiment, the gelatin has a kinematic viscosity of about 24-28 mPa·s, as determined under the following conditions: a gelatin aqueous solution with a concentration of 66.7 g/L and a water bath with a temperature of 60° C., which is determined by a rotary viscometer well-known to those skilled in the art.

As used herein, the term “propylene glycol” refers to a good solvent capable of dissolving many organic substances and inorganic substances, which has been widely used as a solvent in the chemical industry. In cosmetics, the propylene glycol is commonly used as a humectant and a moisturizer to adjust the viscosity and moisturizing properties of products. In the food industry, the propylene glycol reacts with fatty acids to generate propylene glycol fatty acid esters and is primarily used as a food emulsifier. Meanwhile, the propylene glycol is also an excellent solvent for flavorings and pigments. Additionally, the propylene glycol can also be used as a stabilizer, a coagulant, an anti-caking agent, a defoaming agent, an emulsifier, a moisture-retaining agent, and a thickener.

As used herein, the term “hexylene glycol” refers to an organic compound with multiple isomers and generally refers to 1,2-hexanediol unless otherwise specified, which can be used as a matrix ingredient of a topical anesthetic for treating skin inflammation and other diseases, and can be used as a pharmaceutical auxiliary material, etc.

As used herein, the term “maltitol” refers to a compound with various uses and properties, which can be used as an auxiliary material of an inhalation drug to improve the stability, solubility and inhalability of the drug, thereby improving a delivery effect of the drug, and can also be used as an auxiliary material of an anesthetic to facilitate adjustment of the solubility and osmotic pressure of the anesthetic. Meanwhile, the maltitol can be used as a sustained-release agent to control the release rate of a drug in the body, thereby prolonging the action time of the drug.

As used herein, the term “glycerol” refers to a plasticizer commonly used in soft capsule skin, which can improve the flexibility of the capsule skin.

As used herein, the term “sorbitol” refers to a sugar alcohol capable of being slowly metabolized in the human body, which is a polyhydric alcohol formed by reduction of an aldehyde group of glucose and is structurally similar to a monosaccharide. The sorbitol has hygroscopicity, can prevent food from cracking and keep food fresh and soft, and meanwhile, can be used as a pharmaceutical auxiliary material in the field of medicine, such as an adhesive, a humectant and an excipient of a tablet, a capsule and a granule.

As used herein, the term “CGKRK peptide” refers to a polypeptide with an amino acid sequence of CGKRK, which is sequentially composed of five residues including cysteine, glycine, lysine, arginine and lysine. Due to presence of the arginine and the lysine, the peptide has a cationic charge under physiological conditions and is commonly used as a cell-penetrating peptide in the field of biomedicine.

As used herein, “amino acids” refer to amino acids including cysteine, glycine, lysine and arginine. The amino acids are important organic molecules in living organisms and have functions such as participating in construction of cell structures, regulating metabolic processes in the living organisms, and transmitting signals. In the present disclosure, the amino acids are used as an antioxidant.

As used herein, the concept of the term “prevention and/or treatment” indicates any measure suitable for treating deficiencies of vitamins A and D and related diseases, or for performing prophylactic treatment on diseases with such manifestations or symptoms manifested, or for avoiding recurrence of such diseases, such as treating the symptoms of the diseases that recur or have already occurred after the end of a treatment time period, or for preemptively preventing, inhibiting or reducing the occurrence of such diseases or symptoms.

As used herein, the term “plasticizer” refers to a substance capable of increasing the plasticity of a polymer material, which is usually a liquid having a high boiling point and being difficult to volatile or a solid with a low melting point.

As used herein, the term “I.U.” is an abbreviation of international unit. The unit is commonly used in drugs, and is particularly used for representing the content or titer of biological products such as vitamins, hormones, antibiotics, and antitoxins.

In one typical specific embodiment of the present disclosure, use of a CGKRK peptide as an anti-aging agent in preparation of a vitamin A and D soft capsule is provided.

Specifically, the use includes improving aging resistance of a capsule wall material of the vitamin A and D soft capsule to alleviate the problems of soft capsule disintegration, delayed dissolution and a decreased dissolution rate, thereby improving the stability and bioavailability of a vitamin A and D soft capsule preparation.

Raw materials of the capsule wall material of the vitamin A and D soft capsule may include gelatin.

A mass fraction of the CGKRK peptide in the vitamin A and D soft capsule is not greater than 5 wt %. The inventor has found through research that an anti-aging effect of the CGKRK peptide added on the capsule wall material exhibits a first increasing and then decreasing trend along with the addition amount. When the use amount of the CGKRK peptide is 2 wt % or 4 wt %, it has found that the CGKRK peptide added at a relatively higher amount leads to rapid degradation of the capsule wall material and even results in a longer disintegration time in an accelerated test compared with a soft capsule without addition of any antioxidant. Therefore, the mass fraction of the CGKRK peptide in the vitamin A and D soft capsule is not greater than 1 wt %, and is further 0.3-0.8 wt %, such as 0.3 wt %, 0.4 wt %, 0.5 wt %, 0.6 wt %, 0.7 wt %, 0.8 wt %, etc.

In one or more specific embodiments of the present disclosure, a vitamin A and D soft capsule is provided, which includes a capsule wall material and a capsule core medicine liquid, and in parts by weight, a mass ratio of raw materials of the capsule wall material to the capsule core medicine liquid is 1:(1-3), is further 1:(1-2), such as 1:1, 1:1.5, or 1:2, and is particularly preferably 1:1.5.

The raw materials of the capsule wall material include gelatin, a plasticizer, a CGKRK peptide, and water; and the capsule core medicine liquid includes vitamin A, vitamin D, and an oil solvent.

In one or more specific embodiments, the raw materials of the capsule wall material include, in parts by weight:
35-45 parts of the gelatin,
20-30 parts of the plasticizer,
0.3-0.8 part of the CGKRK peptide, and
20-40 parts of the water.

In one or more specific embodiments, the raw materials of the capsule wall material include, in parts by weight:
38-42 parts of the gelatin,
22-28 parts of the plasticizer,
0.3-0.7 part of the CGKRK peptide, and
30-40 parts of the water.

In one specific embodiment, the raw materials of the capsule wall material include, in parts by weight:
40 parts of the gelatin,
25 parts of the plasticizer,
0.5 part of the CGKRK peptide, and
30-40 parts of the water.

In one or more specific embodiments, the plasticizer includes one or more of propylene glycol, glycerol, hexylene glycol, sorbitol, and maltitol, etc., and is preferably a combination of the glycerol and the sorbitol, wherein a mass ratio of the glycerol to the sorbitol is (2-6):1, and is preferably (3-5):1, such as 4:1. The combination of the glycerol and the sorbitol can further reduce the oxygen permeability of the gelatin capsule wall material and lower the aging degree of the capsule wall material.

In one or more specific embodiments, the raw materials of the capsule wall material of the vitamin A and D soft capsule further include, in parts by weight, 0.5-5 parts, preferably 0.5-1 part, and more preferably 2 parts, of polyethylene glycol 1000 vitamin E succinate (TPGS1000, CAS No. 9002-96-4). In the present disclosure, it has found through research that addition of the TPGS1000 into the CGKRK peptide system can enhance aging resistance of the capsule wall material, thereby shortening the disintegration time.

In one or more specific embodiments, in the capsule core medicine liquid, the vitamin A is selected from one or more of vitamin A1, vitamin A2, vitamin A palmitate, and vitamin A acetate, and is preferably the vitamin A acetate; and the vitamin D is vitamin D2 and/or vitamin D3.

In one or more specific embodiments, in the capsule core medicine liquid, only the vitamin A and the vitamin D are contained to serve as active ingredients. In some embodiments, in the capsule core medicine liquid, composite vitamin AD2 or AD3 is contained;
wherein an I.U. ratio of the vitamin A to the vitamin D may be (2-10):1, such as 3:1 or 10:1.

In one or more specific embodiments, in the capsule core medicine liquid, the oil solvent includes an animal oil and a vegetable oil, wherein the animal oil includes, but is not limited to, lard, mutton tallow, beef tallow, cod liver oil, etc; and the vegetable oil includes, but is not limited to, peanut oil, olive oil, soybean oil, castor oil, sunflower seed oil, etc. Preferably, the oil solvent is the soybean oil.

In one or more specific embodiments, a content of the vitamin A in the capsule core medicine liquid may be 1,000-2,000 I.U. of the vitamin A per 0.1-0.5 g of the capsule core medicine liquid. In some embodiments, the capsule core medicine liquid (0.3 g) of each capsule may contain 1,500 I.U. of the vitamin A and 500 I.U. of the vitamin D.

In one or more specific embodiments of the present disclosure, a method for preparing the aforementioned vitamin A and D soft capsule is provided, which includes the following steps:
S1. mixing and dissolving the other components excluding the gelatin and the CGKRK peptide in the raw materials of the capsule wall material under heating to obtain a mixture;
S2. swelling the gelatin in water, adding the swelled gelatin into the mixture prepared in the step S1, then adding the CGKRK peptide, performing even mixing and melting to prepare a gelatin solution while maintaining a heating temperature in the step S1, removing air bubbles in the gelatin solution, and then subjecting the gelatin solution to discharging, filtration and standing for later use;
S3. preparing the gelatin solution prepared in the step S2 into a gelatin strip;
S4. dissolving the vitamins A and D in the oil solvent to prepare the capsule core medicine liquid; and
S5. subjecting an effective amount of the capsule core medicine liquid and the gelatin strip prepared in the step S3 to pelleting to obtain the vitamin A and D soft capsule.

Obviously, in the present disclosure, there is no order of priority between the steps S1-S3 and the step S4, and thus, the preparation method may be implemented in a sequence of S1-S5 or in a sequence of S4, S1-S3 and S5; and alternatively, the step S4 may also be performed while the steps S1-S3 are performed, and then the step S5 is performed.

In one or more specific embodiments, in the step S1, a heating temperature is 60-80° C., such as 70° C.

In one or more specific embodiments, in the step S2, the filtration is performed at 80-120 meshes, and is preferably performed at 100 meshes.

In one or more specific embodiments, in the step S2, a standing time is not less than 2 hours.

In the step S5, the term “effective amount” refers to an amount of active compounds (vitamin A and vitamin D) including the compounds of the present disclosure, wherein the amount is capable of eliciting biological or medical responses of tissue systems, animals, or humans as sought by researchers, veterinarians, physicians, or other medical personnel, and the responses include alleviation or partial alleviation of symptoms of treated diseases, syndromes, conditions, or disorders. In the present disclosure, the soft capsule is particularly used for preventing and/or treating deficiencies of vitamins A and D, such as rickets, nyctalopia, and infantile tetany.

During the pelleting, a temperature of the capsule core medicine liquid is controlled at 20-30° C., such as 25° C.

The present disclosure is further illustrated below in combination with examples. The present disclosure is further illustrated below through the way of examples, but the present disclosure is not limited to the scope of the described examples accordingly. Based on the examples of the present disclosure, any alternations of the present disclosure that are made by those skilled in the art without exerting creative efforts shall fall within the scope of protection of the present disclosure. Meanwhile, in the examples of the present disclosure, unless otherwise specified, all raw materials for preparation are commercially available commodities well-known to those skilled in the art.

### EXAMPLES

In Examples 1-11, vitamin A and D soft capsules containing 0.3 g per capsule of inclusions and 0.2 g per capsule of capsule wall material compositions were prepared. Prescriptions of the capsule wall material compositions are shown in Table 1.

Specific preparation steps of the vitamin A and D soft capsules are as follows.
(1) Material preparation: Various raw materials and auxiliary materials were accurately weighed according to proportions in the prescriptions, and inspection of semi-finished products was performed.
(2) Material mixing: Other ingredients excluding gelatin and a CGKRK peptide or amino acids in the various prescriptions were additionally taken, placed in sol tanks, and heated to 70° C. for dissolution and even mixing.
(3) Gelatin swelling: A prescription amount of the gelatin was taken, and an appropriate amount of water was added to allow the gelatin to absorb the water for swelling.
(4) Preparation of gelatin solutions: The swollen gelatin was added into the sol tanks, then a prescription amount of the CGKRK peptide (purchased from Shanghai Apeptide Biotechnology Co., Ltd.) or prescription amounts of the amino acids (various amino acids purchased from Shanghai Aladdin) were added, stirred and melted, temperatures in the tanks were maintained at about 70° C., and then vacuuming was performed until gelatin solutions had not air bubbles. Then, the gelatin solutions were discharged and filtered at 100 meshes, and filtrates were subjected to heat preservation and standing for more than 2 hours for later use.
(5) Preparation of gelatin strips: The molten gelatin solutions were poured onto a cold drum-shaped cylinder through an automatic rotary capsule press (RGY6-20 soft capsule machine, Beijing Long March Tianmin High-Tech Co., Ltd.) to prepare two frozen gelatin strips with a width of about 5-6 inches.
(6) Preparation of a medicine liquid: Vitamin A acetate and vitamin D3 were separately dissolved in soybean oil and evenly stirred to serve as inclusions for later use, wherein contents of the vitamin A acetate and the vitamin D3 were 1,500 I.U./0.3 g and 500 I.U./0.3 g, respectively.
(7) Pelleting: The frozen gelatin strips were allowed to pass through rollers to provide appropriate strip alignments, and then, the vitamin A and D capsule core medicine liquid was loaded into the automatic rotary capsule press. A temperature of the capsule core medicine liquid was controlled at about 25° C., and pelleting was performed.
(8) Shaping, rinsing and drying: Soft capsules were pressed, shaped and washed with 95% ethanol. The soft capsules were spin-dried and then entered a dryer of the automatic rotary capsule press for drying for 3-5 hours after the ethanol was volatilized.
(9) Capsule selection: The dried soft capsules were selected to pick out the vitamin A and D soft capsules with qualified appearance.

TABLE 1
Composition of different capsule wall material compositions (in parts by weight)

| Example | | 1 | 2 | 3 | 4 | 5 | 6 | 7 | 8 | 9 | 10 | 11 |
|---|---|---|---|---|---|---|---|---|---|---|---|---|
| Gelatin | — | 40 | 40 | 40 | 40 | 40 | 40 | 40 | 40 | 40 | 40 | 40 |
| Plasticizer | Glycerol | 20 | 20 | 20 | 20 | 20 | 20 | 20 | 20 | 20 | 20 | 20 |
| | Sorbitol | 5 | 5 | 5 | 5 | 5 | 5 | 5 | 5 | 5 | 5 | 5 |
| Antioxidant | CGKRK | 0.5 | 0.5 | 0.3 | 0.8 | 2 | 4 | | | | | |
| | peptide | | | | | | | | | | | |
| | Cysteine | | | | | | | 2 | | | | |
| | Glycine | | | | | | | | 2 | | | |
| | Lysine | | | | | | | | | 2 | | |
| | Arginine | | | | | | | | | | 2 | |
| | TPGS- | 2 | | | | | | | | | | |
| | PEG1000 | | | | | | | | | | | |
| Water | — | Balance | Balance | Balance | Balance | Balance | Balance | Balance | Balance | Balance | Balance | Balance |
| Total | | 100 | 100 | 100 | 100 | 100 | 100 | 100 | 100 | 100 | 100 | 100 |

### Content Determination

The qualified vitamin A and D soft capsules prepared in various examples were determined in accordance with a content determination method for a vitamin A and D soft capsule in the “Pharmacopoeia of the People's Republic of China (2020 Edition)”.

### Dissolution Determination

A disintegration time test was carried out on the qualified vitamin A and D soft capsules prepared in various examples. Specifically, a second method (paddle method) of a dissolution rate determination method in the “Pharmacopoeia of the People's Republic of China (2020 Edition)” was adopted. The vitamin A and D soft capsules were taken and determined at a speed of 50 r/min with 900 mL of purified water as a dissolution medium.

### Accelerated Test

The vitamin A and D soft capsules prepared in various examples were packaged in polyester/aluminum/polyethylene pharmaceutical composite films and then subjected to an accelerated test under the conditions of a temperature of 40° C. and a relative humidity of 75%. After lasting for 3 or 6 months, samples were taken for content determination and disintegration time detection. Since an aging degree of a capsule wall material of a soft capsule is positively correlated with a disintegration time, the disintegration time of the soft capsule is used as an inspection index to assess the aging degree.

Test results are shown in Tables 2-3.

TABLE 2
Contents and disintegration times of vitamin A and D
soft capsules prepared in various examples at 0 month

| | 0 month | | |
|---|---|---|---|
| | Vitamin A acetate | Vitamin D₃ | Disintegration time |
| Example | (I.U.) | (I.U.) | (min) |
| 1 | 1482 | 486 | 6.0 |
| 2 | 1469 | 475 | 6.2 |
| 3 | 1476 | 468 | 6.3 |
| 4 | 1462 | 482 | 6.6 |
| 5 | 1456 | 467 | 7.4 |
| 6 | 1468 | 484 | 8.2 |
| 7 | 1483 | 472 | 6.4 |
| 8 | 1466 | 466 | 6.8 |
| 9 | 1435 | 487 | 6.4 |
| 10 | 1464 | 475 | 6.5 |
| 11 | 1436 | 484 | 5.6 |

TABLE 3
Contents and disintegration times of vitamin A and D soft capsules
prepared in various examples after acceleration for 3 and 6 months

| | 3 months | | | 6 months | | |
|---|---|---|---|---|---|---|
| | Vitamin A | Vitamin | Disintegration | Vitamin A | Vitamin | Disintegration |
| | acetate | D₃ | time | acetate | D₃ | time |
| Example | (%) | (%) | (min) | (%) | (%) | (min) |
| 1 | 98.5 | 98.4 | 7.5 | 98.1 | 98.2 | 8.1 |
| 2 | 97.4 | 98.0 | 7.9 | 97.5 | 97.5 | 8.8 |
| 3 | 97.4 | 97.5 | 8.5 | 97.1 | 97.3 | 10.4 |
| 4 | 97.5 | 97.8 | 9.2 | 97.2 | 97.4 | 11.3 |
| 5 | 93.1 | 92.6 | 37.4 | 89.4 | 84.1 | 46.9 |
| 6 | 90.4 | 87.8 | 40.6 | 84.2 | 81.3 | 51.8 |
| 7 | 95.8 | 92.6 | 14.5 | 90.3 | 87.5 | 21.5 |
| 8 | 94.2 | 90.1 | 17.6 | 87.5 | 85.3 | 26.1 |
| 9 | 93.5 | 89.3 | 15.7 | 87.4 | 83.0 | 23.8 |
| 10 | 90.4 | 88.6 | 18.5 | 81.9 | 78.6 | 29.4 |
| 11 | 89.5 | 85.8 | 32.6 | 80.5 | 78.5 | 39.5 |

### Result Analysis

Through comparison, the vitamin A and D soft capsule prepared without addition of any antioxidant (Example 11) and the soft capsules obtained by adding reportedly effective different amino acids (cysteine, glycine, lysine and arginine) and the CGKRK peptide as an antioxidant (Examples 2-10) have great differences in disintegration time and content results in the accelerated test.

The disintegration time of the vitamin A and D soft capsule prepared without addition of any antioxidant is prolonged from 5.6 min to 32.6 min in the accelerated test for 3 months, and is further prolonged to 39.5 min in the accelerated test for 6 months, indicating that the capsule wall material of the soft capsule has severe aging, and the contents of the vitamins A and D in the soft capsule are also obviously decreased.

In Examples 7-10, 2 wt % of the cysteine, the glycine, the lysine or the arginine is respectively added into the capsule wall material, and the disintegration time of the prepared vitamin A and D soft capsules is shortened to varying degrees in disintegration time tests for 3 months and 6 months compared with a microcapsule without addition of any antioxidant, indicating that these amino acids play a certain degree of an anti-aging effect on the capsule wall material, and the contents of the vitamins A and D in the soft capsules are also increased.

In Examples 2-4, 0.3-0.8 wt % of the CGKRK peptide is added, the disintegration time of the prepared corresponding vitamin A and D soft capsules is about 6 min at 0 month, the disintegration time is increased to 7.9-9.2 min after acceleration for 3 months, and the disintegration time is further increased to 8.8-11.3 min after 6 months, indicating a more significant anti-aging advantage compared with the soft capsules without addition of an antioxidant or with addition of the amino acids. It is worth noting that an addition amount of the CGKRK peptide with a significant anti-aging effect is obviously decreased compared with addition amounts of other amino acids, further indicating that the CGKRK peptide plays an unexpected anti-aging effect on the capsule wall material with the gelatin as a matrix. The reasons are that since the CGKRK peptide simultaneously contains active groups such as free sulfydryl, amino and guanidyl, the peptide provides a more complex antioxidant or anti-crosslinking pathway and competitively captures reactive oxygen species in the capsule wall material system or has a higher ability to competitively bind to aldehyde molecules from external sources, thereby avoiding damage to a gelatin matrix structure in the capsule wall material and showing a superior anti-aging effect compared with single amino acids.

Further, an effect of the addition amount of the CGKRK peptide on the capsule wall material is studied. From comparison between Examples 2-4, it can be found that after the accelerated test for 6 months, the disintegration time of the soft capsule added with 0.5 wt % of the CGKRK peptide is prolonged by 46.7% compared with that at 0 month, the disintegration time of the soft capsule added with 0.3 wt % of the CGKRK peptide is prolonged by 65.1% compared with that at 0 month, and the disintegration time of the soft capsule added with 0.8 wt % of the CGKRK peptide is prolonged by 71.2% compared with that at 0 month, indicating to a certain degree that the anti-aging effect of the CGKRK peptide added on the capsule wall material exhibits a first increasing and then decreasing trend along with the addition amount. When the use amount of the CGKRK peptide is further increased to 2 wt % and 4 wt % (Examples 5-6), the inventor finds that the CGKRK peptide added at a relatively higher amount leads to rapid deterioration of the capsule wall material and even results in a longer disintegration time in the accelerated test compared with the soft capsule without addition of any antioxidant. The reason may be that since the lysine, arginine and cysteine residues in the excessive CGKRK peptide undergo crosslinking with groups in the gelatin under high-temperature and high-humidity conditions of the accelerated test, aging of the gelatin in the capsule wall material is accelerated.

When 2 wt % of TPGS-PEG1000 is further added into the system added with 0.5 wt % of the CGKRK peptide, the inventor finds that the PGS-PEG1000 added slightly shortens the disintegration time at 0 month, 3 months and 6 months, which may be related to the presence of a PEG chain and vitamin E in the TPGS-PEG1000, thereby acting together with the CGKRK peptide to enhance the anti-aging effect on the capsule wall material.

It should be noted that the above examples are only used to illustrate the technical solutions of the present disclosure and are not intended to limit the present disclosure. Although the present disclosure has been illustrated in detail with reference to the provided examples, those skilled in the art can make modifications or equivalent substitutions to the technical solutions of the present disclosure as needed without departing from the spirit and scope of the technical solutions of the present disclosure.

1. A vitamin A and D soft capsule, comprising a capsule wall material and a capsule core medicine liquid, wherein in parts by weight, a mass ratio of raw materials of the capsule wall material to the capsule core medicine liquid is 1:(1-3);
wherein the raw materials of the capsule wall material comprise gelatin, a plasticizer, a CGKRK peptide, and water; and the capsule core medicine liquid comprises vitamin A, vitamin D, and an oil solvent.

2. The vitamin A and D soft capsule according to claim 1, wherein the mass ratio of the capsule wall material to the capsule core medicine liquid is 1:(1-2).

3. The vitamin A and D soft capsule according to claim 1, wherein a mass fraction of the CGKRK peptide in the vitamin A and D soft capsule is not greater than 5 wt %.

4. The vitamin A and D soft capsule according to claim 1, wherein the raw materials of the capsule wall material comprise, in parts by weight:
35-45 parts of gelatin,
20-30 parts of the plasticizer,
0.3-0.8 part of the CGKRK peptide, and
20-40 parts of water.

5. The vitamin A and D soft capsule according to claim 1, wherein the raw materials of the capsule wall material comprise, in parts by weight:
38-42 parts of gelatin,
22-28 parts of the plasticizer,
0.3-0.7 part of the CGKRK peptide, and
30-40 parts of water.

6. The vitamin A and D soft capsule according to claim 1, wherein the plasticizer comprises one or more selected from the group consisting of propylene glycol, glycerol, hexylene glycol, sorbitol, and maltitol.

7. The vitamin A and D soft capsule according to claim 6, wherein the plasticizer is a combination of glycerol and sorbitol, and a mass ratio of glycerol to sorbitol is (2-6):1.

8. The vitamin A and D soft capsule according to claim 1, wherein the raw materials of the capsule wall material of the vitamin A and D soft capsule further comprise, in parts by weight, 0.5-5 parts of polyethylene glycol 1000 vitamin E succinate.

9. The vitamin A and D soft capsule according to claim 1, wherein the raw materials of the capsule wall material of the vitamin A and D soft capsule further comprise, in parts by weight, 0.5-1 parts of polyethylene glycol 1000 vitamin E succinate.

10. The vitamin A and D soft capsule according to claim 1, wherein in the capsule core medicine liquid, the vitamin A is selected from one or more of vitamin Al, vitamin A2, vitamin A palmitate, and vitamin A acetate.

11. The vitamin A and D soft capsule according to claim 1, wherein the vitamin A is vitamin A acetate; and the vitamin D is vitamin D2 and/or vitamin D3.

12. The vitamin A and D soft capsule according to claim 1, wherein the capsule core medicine liquid contains only vitamin A and vitamin D as active ingredients.

13. The vitamin A and D soft capsule according to claim 1, wherein an I.U. ratio of vitamin A to vitamin D is (2-10):1.

14. The vitamin A and D soft capsule according to claim 1, wherein the capsule core medicine liquid contains composite vitamin AD2 or AD3.

15. The vitamin A and D soft capsule according to claim 1, wherein the oil solvent comprises one or more selected from an animal oil and a vegetable oil, wherein the animal oil comprises lard, mutton tallow, beef tallow, and cod liver oil; the vegetable oil comprises peanut oil, olive oil, soybean oil, castor oil, and sunflower seed oil.

16. The vitamin A and D soft capsule according to claim 1, wherein the oil solvent is soybean oil.

17. The vitamin A and D soft capsule according to claim 1, wherein a content of vitamin A in the capsule core medicine liquid is 1,000-2,000 I.U. of vitamin A per 0.1-0.5 g of the capsule core medicine liquid.

18. A method for preparing the vitamin A and D soft capsule according to claim 1, comprising the following steps:
S1. mixing and dissolving other components excluding gelatin and the CGKRK peptide in raw materials of a capsule wall material under heating to obtain a mixture;
S2. swelling gelatin in water, adding the swelled gelatin into the mixture prepared in step S1, then adding the CGKRK peptide, performing even mixing and melting to prepare a gelatin solution while maintaining a heating temperature in step S1, removing air bubbles in the gelatin solution, and then subjecting the gelatin solution to discharging, filtration and standing for later use;
S3. preparing the gelatin solution prepared in step S2 into a gelatin strip;
S4. dissolving vitamins A and D in an oil solvent to prepare a capsule core medicine liquid; and
S5. subjecting an effective amount of the capsule core medicine liquid and the gelatin strip prepared in step S3 to pelleting to obtain the vitamin A and D soft capsule.

19. The method according to claim 18, wherein there is no order of priority between the steps S1-S3 and the step S4, and thus, the method is implemented in a sequence of S1-S5 or in a sequence of S4, S1-S3 and S5; or alternatively, step S4 is performed simultaneously with steps S1-S3, and then step S5 is performed.

20. The method according to claim 18, wherein in step S1, a heating temperature is 60-80° C.;
in step S2, the filtration is performed at 80-120 meshes, and a standing time is not less than 2 hours; and
in step S5, during the pelleting, a temperature of the capsule core medicine liquid is controlled at 20-30° C.
